(19) United States
(12) Patent Application Publication
(10) Pub. No.: US 2006/0122150 A1
(43) Pub. Date: Jun. 8, 2006

(54) Pharmaceutical composition and method for treating a joint-capsule arthropathy

(76) Inventors: Dennis C. Argentieri, Milford, NJ (US); Demetrius Carter, Mount Laurel, NJ (US); Laura J. Brown, Hamilton Square, NJ (US); Jeffrey C. Geesin, Doylestown, PA (US); John J. Siekierka, Towaco, NJ (US); Han Cui, Bridgewater, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/239,725

(22) Filed: Sep. 30, 2005

Related U.S. Application Data
(60) Provisional application No. 60/614,579, filed on Sep. 30, 2004.

(51) Int. Cl. A61K 31/728

(52) U.S. Cl. 514/54

(57) ABSTRACT

A pharmaceutical composition for use in treating a joint-capsule arthropathy comprising an effective amount of one or more of a locally administered, optionally encapsulated therapeutic agent in admixture with a hyaluronic acid delivery vehicle and a method for use thereof in treating a joint-capsule arthropathy by intra-articular injection.

### CROSS REFERENCE TO RELATED APPLICATIONS

This present application claims benefit of U.S. Provisional Patent Application Ser. No. 60/614,579, filed Sep. 30, 2004, which is incorporated herein by reference in its entirety and for all purposes.

### FIELD OF THE INVENTION

The present invention is directed to a pharmaceutical composition and a method for use thereof in treating a joint-capsule arthropathy. More particularly, the pharmaceutical composition is an effective amount of one or more of a locally administered, optionally encapsulated therapeutic agent in admixture with a hyaluronic acid delivery vehicle for use in treating a joint-capsule arthropathy by intra-articular injection.

### BACKGROUND OF THE INVENTION

Joint-capsule arthropathy is a complex, progressive disease characterized by degeneration of the joint-capsule cartilage, hypertrophy of bone at the margins of the motion segment and changes in the synovial membrane.

As the arthropathy progresses, the mechanical and biochemical interplay between the tissues in the joint capsule (bone, synovial tissue and cartilage) changes, resulting in synovial inflammation, elevated pro-inflammatory mediator levels, cartilage destruction and severe pain.

Cartilage is a connective tissue that is produced by chondrocyte cells embedded in an extracellular matrix (ECM). The ECM is composed of collagen type II in the form of fine fibers and an amorphous material having large amounts of proteoglycan. The amorphous material additionally contains plasma constituents, metabolites, water and ions found between the chondrocyte cells and collagen fibers. Proteoglycans are important in determining the viscoelastic properties of joints and contain functional groups that retain water, thereby providing the cartilage with its lubricating qualities. The proteoglycans also secrete small amounts of cytokines (such as IL-1β, TNF-α and IL-6) and matrix metalloproteinases (MMPs) that help regulate the metabolism of the chondrocytes. The core of complex proteoglycan aggregates found in the ECM are formed by hyaluronic acid (HA), a high molecular weight polymer composed of repeating dimeric units of glucuronic acid and N-acetyl glucosamine.

Pharmaceutical compositions and methods of treatment known to be effective in treating arthropathies have contained HA as the active ingredient. Use of HA as an arthropathic therapy provides temporarily relief of chronic symptoms such as joint pain and stiffness as a result of its viscosupplementation properties. However, use of HA as the sole active agent for treating an arthropathy does not directly relieve chronic symptoms or modify the progression of the arthropathic disease.

Inflammatory pathways mediate the etiology of progressive, degenerative, arthropathies. Therefore, an arthropathic disease may be modified by disrupting the inflammatory cascade using an anti-inflammatory agent, e.g., inhibiting the 5LO-1, COX-1, COX-2, LTB₄ or TXB₂ pathways. Accordingly, anti-inflammatory agents have been widely used for treating arthropathic symptoms by various administration routes such as orally, intravenously or by subcutaneous or intramuscular injection. Such methods of administration deliver the agent systemically, without specifically targeting treatment at the site of inflammation or modifying the progression of the disease.

A common problem with NSAIDs delivered systemically is the disruption of COX 1. COX 1 is continuously stimulated by the body to produce prostaglandins that are involved with normal daily functions (digestion, kidney function and platelet production). Chronic, systemic administration of anti-inflammatory agents unnecessarily distributes a large amount of the agent over the whole body in order to provide therapeutic benefit at the site of inflammation. Systemic administration unnecessarily affects body systems and organs that have no need of treatment and unnecessarily subjects those already suffering to further unpleasant and debilitating side effects. Moreover, since arthropathies require chronic treatment, the long-term dosing regiments often result in adverse effects. These effects may include, diarrhea, nausea and ulceration of the gastrointestinal system as a cost for their therapeutic benefit.

Accordingly, there remains a need for pharmaceutical compositions and methods of treatment effective for use in treating joint-capsule arthropathies.

U.S. Pat. No. 4,937,254 (Sheffield) describes a method for inhibiting post-surgical adhesion formation by topical locally effective administration of a NSAID active ingredient in a polymeric microcapsule formulation to the injured tissue. Sheffield also refers to U.S. Pat. No. 4,522,803 (Lenk) as particularly describing phospholipid vesicles containing steroid anti-inflammatory agents, to U.S. Pat. No. 4,427,649 (Dingle) as describing phospholipid vesicles (liposomes) containing steroid anti-inflammatory agents for the treatment of rheumatoid arthritis, to U.S. Pat. No. 4,389,330 (Tice) as describing the microencapsulation of pharmaceutically active agents in various materials, including lactide and glycolide polymers, to U.S. Pat. No. 3,755,558 (Scribner) as describing polylactide-drug mixtures for topical application, including anti-inflammatory drugs such as hydrocortisone, to U.S. Pat. No. 4,517,295 (Bracke) as describing the possible use of hyaluronic acid to treat post-surgical adhesions and to U.S. Pat. No. 4,141,973 (Balazs) as describing the use of certain ultra-pure, high molecular weight fractions of hyaluronic acid for use in anti-adhesion.

U.S. Pat. No. 5,095,037 (Iwamitsu) describes a pharmaceutical composition for treating inflammatory disorders comprising (a) an effective amount of hyaluronic acid or its salt, and (b) an effective amount of an anti-inflammatory agent such as diclofenac or ibuprofen in a combination ratio for administration into an articular cavity.

European Patent EP1153607A2 (Dunn) describes a method for preparing a joint prior to treatment for inflammation by injecting or otherwise applying a group of agents such as anti-cytokines, anti-kinases, anti growth factors individually or in various combinations thereof, then treating the joint by injecting a mixture of a purified growth hormone such as somatotrophin and a buffer solution into the joint.

PCT Application WO03000190A2 (Thompson) describes a pharmaceutical composition for the treatment of arthritic joints such as in osteoarthritis by intraarticular injection comprising a liposomally encapsulated glycosaminoglycan (GAG) such as hyaluronic acid administered either singly or in combination with another agent such as a non-steroidal anti-inflammatory drug (NSAID), a p38 kinase inhibitors, a TNF-α inhibitor, a corticosteroid, an enzyme inhibitor such as a hyaluronidase inhibitor, a MMP inhibitor, an aggrecanase inhibitor, an apoptosis inhibitor such as EPO, a cartilage enhancing factor such as TGF-131 or a Bone Morphogenic Protein (BMP), wherein the other agent is either optionally co-encapsulated with the GAG, bound to the liposome but not encapsulated, or present as free drug outside the liposome.

### SUMMARY OF THE INVENTION

The present invention is directed to a pharmaceutical composition for use in treating a joint-capsule arthropathy.

The present invention is further directed to a pharmaceutical composition for use in treating a joint-capsule arthropathy comprising an effective amount of one or more of a locally administered, optionally encapsulated therapeutic agent in admixture with a hyaluronic acid delivery vehicle.

An embodiment of the pharmaceutical composition comprises at least one encapsulated therapeutic agent in the admixture.

Another embodiment of the pharmaceutical composition comprises one or more of an encapsulated therapeutic agent and one or more of an additional, different optionally encapsulated therapeutic agent in the admixture.

Another embodiment of the pharmaceutical composition comprises one or more of an encapsulated therapeutic agent and one or more of an additional, different unencapsulated therapeutic agent in the admixture.

Another embodiment of the pharmaceutical composition comprises two or more of an encapsulated therapeutic agent in the admixture, wherein each encapsulated agent has a dissimilar release profile.

Another embodiment of the pharmaceutical composition comprises two or more of an encapsulated therapeutic agent, each agent having a dissimilar release profile, and one or more of an optionally encapsulated therapeutic agent in the admixture.

An embodiment of the therapeutic agent for use in the present invention is one or more of a non-steroidal anti-inflammatory drug (NSAID).

An embodiment of the composition includes two or more of a therapeutic agent, wherein at least one is an NSAID in combination with at least one other therapeutic agent.

The present invention is also directed to a method for treating a joint-capsule arthropathy in a subject in need thereof comprising locally administering to the subject of an effective amount of a pharmaceutical composition comprising an effective amount of one or more of an optionally encapsulated therapeutic agent in admixture with a hyaluronic acid delivery vehicle.

The effective amount of the pharmaceutical composition for use in the method of the present invention is a non-systemic, therapeutically effective amount.

An embodiment of the method of locally administering the composition further comprises transcapsular or transdiscal administration for treating a joint-capsule arthropathy.

An embodiment of the method comprises administering at least one encapsulated therapeutic agent in the admixture.

Another embodiment of the method comprises administering one or more encapsulated therapeutic agent and one or more additional, different optionally unencapsulated therapeutic agent in the admixture.

Another embodiment of the method comprises administering two or more of an encapsulated therapeutic agent in the admixture, wherein each encapsulated agent has a dissimilar release profile.

Another embodiment of the method comprises administering two or more of an encapsulated therapeutic agent, each having a dissimilar release profile, and one or more unencapsulated therapeutic agent in the admixture.

The pharmaceutical composition and method of the present invention have the benefit of efficiency and suitability for both short and long-term, as well as acute and chronic administration and disease modification.

### DETAILED DESCRIPTION

The present invention is directed to a pharmaceutical composition for use in treating a joint-capsule arthropathy.

The particular joint-capsules of the human skeletal system contemplated for treatment in the intended scope for use of the present invention include, without limitation, a relatively intact joint-capsule formed by opposing bone surfaces having respective opposing hyaline cartilage articular surfaces, having a peripheral, collagenous ligamentous capsule connecting the articular surfaces thereby defining a central joint space, having a synovium lining upon an inner wall of the capsule, and synovial fluid contained within the joint space.

More particularly, the scope includes joint-capsules that are subject to wear, repetitive motion or immobility and the like and the consequent presence of pro-inflammatory molecules resulting therefrom selected from those related to the hip, knee, shoulder, ankle, elbow, wrist, toe, finger or spine. Joint-capsule spaces related to the spine include the disc-capsule nucleus pulposus and annulus fibrosus spaces and associated opposing spinal facet joint surfaces.

“Spinal facet joint” means those posterior elements of the spine that help to support axial, torsional and shear loads that act on the spinal column. Furthermore, the facet joints are diarthroidal joints that provide both sliding articulation and load transmission features. The diarticular surfaces of the facets contact in extension, limiting rotation and increasing compressive load. The diarticular surfaces also contact on one side of the spine during lateral bending and axial rotation, thereby limiting rotation and transferring load.

“Relatively intact” means that the integrity of the joint-capsule space is degenerating but has not yet degenerated to the extent that the method of the present invention would be ineffective.

The particular joint-capsule arthropathy contemplated for treatment by use of the present invention includes, without limitation, osteoarthritis, joint-capsule injuries which have triggered or will trigger a pro-inflammatory response or arthropathies which have triggered or will trigger a pro-inflammatory response.

Joint-capsule injury etiology includes, without limitation, injuries resulting from endogenous sources or exogenous sources of insult.

Joint-capsule injury includes, without limitation, injuries resulting from endogenous sources of insult such as a genetic joint-disorder, abnormal bone growth, aggregation or cyst formation in a joint space, joint fusion, joint misalignment, compensation for joint misalignment or other types of musculo-skeletal injuries in other parts of the body or injuries resulting from wear, repetitive motion or immobility and the like.

Joint-capsule injury includes, without limitation, injuries resulting from exogenous sources of insult such as a musculo-skeletal injury caused by trauma, sport-related injuries or immobility and the like.

Accordingly, the scope of joint-capsule arthropathies that may be treated by the instant pharmaceutical composition are selected from a symptomatic range of arthropathies that are just beginning to develop, where bone degeneration and the level of pain experienced by the subject is at a minimum to those arthropathies wherein bone degeneration and the level of pain experienced by the subject requires surgical intervention such as joint replacement or where the administration of one or more of a therapeutic agent for pain intervention has led to a decreased quality of life.

The composition of this invention may be locally administered to provide relief from pain, to pharmacodynamically modify the joint-capsule arthropathy and the progression of the disease or to delay surgical intervention.

The particular joint-capsule injury contemplated for treatment in the intended scope of use for the present invention includes, without limitation, injured joint-capsules which will or which presently have an arthropathy or injured joint-capsules which presently have an arthropathy which has triggered or will trigger a pro-inflammatory response.

The pharmaceutical composition of the present invention is a combination product comprising one or more of an optionally encapsulated therapeutic agent incorporated in a hyaluronic acid delivery vehicle, wherein the agent is one or more compounds or compositions thereof which are capable of reducing inflammation in a joint-capsule arthropathy, improving chronic symptoms such as pain and stiffness and modifying progression of the disease in the joint-capsule.

The ability to determine whether a joint-capsule has an injury originating from a particular source that will result in or has presently has resulted in an arthropathy or whether a joint-capsule has an arthropathy which has triggered or will trigger a pro-inflammatory response is within the skill of those with ordinary experience in the art.

The therapeutic agent for use in the present invention is selected from a NSAD, a nitric oxide (NO) inhibitor, a cyclooxygenase (COX) inhibitor, a prostaglandin (PG) inhibitor, an interleukin inhibitor, a leukotriene (LT) inhibitor, an inflammatory or growth proliferation kinase inhibitor, an inflammatory cytokine inhibitor, a corticosteroid, a hyaluronidase enzyme inhibitor, a MMP inhibitor, an aggrecanase inhibitor, an apoptosis inhibitor, a cartilage enhancing factor or a BMP in admixture with the HA delivery vehicle.

Examples of a NSAID for use in the present invention are selected from analogs of carboxylic acid, acetic acid, propionic acid, salicylic acid, hydratropic acid (such as suprofen), hydroxamic acid (such as tepoxalin), benzoic/xylylanthranilic acid (such as mefenamic acid), benzene acetic acid (such as ibuprofen, diclofenac or alclofenac), indole acetic acid (such as indomethacin), indene acetic acid (such as sulindac), toluoylpyrrole acetic acid (such as tolmetin), naphthalene acetic acid (such as naproxen), benzopyranopyridine acetic acid (such as pranoprofen), pyrazolidinedione (such as phenylbutazone or oxyphenbutazone) or benzthiazine (such as piroxicam) and the like or a salt or ester form or mixture thereof.

Embodiments of the present invention include a NSAID such as suprofen, tepoxalin, ibuprofen, diclofenac, indomethacin, sulindac, tolmetin, naproxen, oxyphenbutazone or a salt or ester form or mixture thereof.

An embodiment of the present invention includes a NSAID selected from suprofen, tolmetin or tepoxalin and the like or a salt or ester form or mixture thereof.

Embodiments of the present invention further include at least one NSAID wherein the NSAID is a cyclooxygenase, leukotriene or prostaglandin inhibitor, wherein the cyclooxygenase inhibitor is selected from a 5LO-1, COX-1 or COX-2 inhibitor and the like, wherein the leukotriene inhibitor is a LTB₄ inhibitor and the like, and wherein the prostaglandin inhibitor is a PGE₂ inhibitor and the like.

Embodiments of the invention include at least one NSAID wherein the NSAID is a 5LO-1, COX-1, COX-2, LTB₄ and TXB₂ inhibitor.

An embodiment of the invention includes an NSAID wherein the NSAID is a 5LO-1 inhibitor having an IC₅₀ of from about 0.010 μM to about 10 μM.

An embodiment of the invention includes an NSAID wherein the NSAID is a COX-1 inhibitor having an IC₅₀ of from about 0.06 μM to about 12 μM.

An embodiment of the invention includes an NSAID wherein the NSAID is a COX-2 inhibitor having an IC₅₀ of from about 0.05 μM to about 6 μM.

An embodiment of the invention includes an NSAID wherein the NSAD is a LTB₄ inhibitor having an IC₅₀ of from about 0.035 μM to about 2 μM.

An embodiment of the invention includes an NSAID wherein the NSAID is a TXB₂ inhibitor having an IC₅₀ of from about 0.002 μM to about 0.04 μM.

Certain embodiments of the invention further include at least one NSAID wherein the NSAID is selected from suprofen, tepoxalin or tolmetin.

Embodiments of the present invention include an inflammatory kinase inhibitor such as a MAP kinase inhibitor, wherein the MAP kinase inhibitor is a p38, ERK or JNK2 kinase inhibitor or an isoform thereof.

An example of a p38 MAP kinase inhibitor isoform is a selective p38α MAP kinase inhibitor.

Embodiments of the present invention include a growth proliferation kinase inhibitor such as sirolimus (rapamycin).

Embodiments of the present invention include an inflammatory cytokine inhibitor such as a TNF-α inhibitor, an IL-1α inhibitor, IL-β inhibitor, IL-6 inhibitor, IL-10 inhibitor, IL-12 inhibitor or an IL-17 inhibitor.

Certain embodiments of the invention an inflammatory cytokine inhibitor selected from a TNF-α inhibitor, an IL-1α inhibitor, IL-1β inhibitor, IL-6 inhibitor or IL-10 inhibitor.

Embodiments of the present invention include a pharmaceutical composition further comprising a combination product having two or more of a foregoing optionally encapsulated therapeutic agent in admixture with the HA delivery vehicle.

Embodiments of a combination product further comprise one or more of an optionally encapsulated therapeutic agent and one or more of an additional, different optionally encapsulated therapeutic agent for use in treating a joint-capsule arthropathy.

Further embodiments of a combination product may include a “cocktail” of an optionally encapsulated therapeutic agents such as, but not otherwise limited to, a mixture of two or more of a therapeutic agent for use in treating a joint-capsule arthropathy, wherein the combination product achieves the desired effect such as pain relief, modification of the arthropathy and its progression or delay of surgical intervention and the like.

Pharmaceutical compositions according to the invention may, alternatively or in addition to an optionally encapsulated therapeutic agent, comprise an optionally encapsulated pharmaceutically acceptable salt of the agent or a pro-drug or pharmaceutically active metabolite of such agent or salt in admixture with a pharmaceutically acceptable carrier.

The term “composition” therefore means a product comprising one or more of an optionally encapsulated therapeutic agent and a pharmaceutically acceptable carrier, or any such alternatives to such an optionally encapsulated agent and a pharmaceutically acceptable carrier, as well as any product, which results, directly or indirectly, from such combinations.

The term “pharmaceutically acceptable” refers to molecular entities and components used in the composition described herein, which are of sufficient purity and quality such that, when appropriately administered to a subject, animal or a human, the composition does not produce an adverse, allergic, or other untoward reaction. Accordingly, either pharmaceutically acceptable compositions or medicaments for either human use (clinical and over-the-counter) or veterinary use are equally included within the scope of the present invention.

“Medicament” refers to one or more compositions of the present invention used in a product for use in treating a joint-capsule arthropathy by intra-articular injection.

The dosages of each agent in the combination product are adjusted when combined to achieve the desired therapeutic effect. As those skilled in the art will appreciate, dosages of each agent may be independently optimized and combined to achieve a synergistic result whereby the pathology is reduced more than it would be if either agent were used alone.

Administration regimens for treating a joint-capsule arthropathy using a combination product of the present invention includes, without limitation, co-administration of one or more of an optionally encapsulated therapeutic agent or mixture of an encapsulated or unencapsulated agent, sequential administration of each such agent, administration of a single composition containing such agent or mixture thereof or simultaneous administration of separate, divided compositions containing each agent. The instant invention is therefore to be understood as embracing all such regimes of simultaneous or alternating treatment and the term “administering” is to be interpreted accordingly.

Examples of the present invention include a product comprising one or more of an optionally encapsulated NSAID.

Examples of the present invention include a product comprising at least one encapsulated NSAID.

Examples include a product comprising one or more of an encapsulated NSAID and one or more of an additional, different therapeutic agent. For such a combination product, one or more of the other agent used in combination with the encapsulated NSAID may optionally be encapsulated.

Examples include a product comprising one or more of an encapsulated NSAID and one or more of an additional, different unencapsulated therapeutic agent.

Examples include a product comprising two or more of an encapsulated NSAID, wherein each encapsulated NSAID has a dissimilar release profile.

Examples include a product comprising two or more of an encapsulated NSAID, wherein each encapsulated NSAID has a dissimilar release profile, and one or more of an optionally encapsulated therapeutic agent.

An encapsulated agent that has been locally administered has an increased residence time in the joint-capsule. Accordingly, such an agent mediates the inflammatory response inside the joint-capsule over a period of time by controlling the release of the agent, thus enabling the ability to provide long-term, chronic administration and disease modification.

A combination product having at least one additional, different, unencapsulated therapeutic agent has the ability to provide a short-term, chronic administration and disease modification. For such a combination product, one or more of the other agent used in combination with the encapsulated therapeutic agent may optionally be encapsulated.

The intra-articular injection of the pharmaceutical composition also functions as a depot administration of the therapeutic agent at a higher dose than could be administered systemically and provides a controlled, sustained release of the agent at the site of inflammation resulting in extended pain relief. Accordingly, the pharmacokinetic profile of the instant pharmaceutical composition provides an opportunity for disease modification, whereby the encapsulated therapeutic agent resides in the joint space and is released over a period of time, thus allowing the agent to disrupt the localized inflammatory response and to be taken up by the cartilage.

While an intra-articular injection of a particulate biomaterial preferentially activates macrophages when the injected biomaterial is in a particle size range of from about 50 microns to about 150 microns, a discovery of the present invention is that an intra-articular injection of a therapeutic microparticle is surprisingly well tolerated.

Embodiments of the present invention include an unencapsulated therapeutic agent in a particle form, wherein the particle form is selected from a finely divided particle, having a particle size in the range of from about 0.5 microns to about 200 microns. Other embodiments include a particle size for the agent in the range of from about 5 microns to about 50 microns. Still other embodiments include a mean particle size for the agent in the range of from about 25 microns to about 50 microns.

One skilled in the art will recognize that the optimal particle size of an agent contemplated for use within the scope of the present invention will vary depending on the size of the small molecule used as the agent and the physical characteristics of the agent when present in bulk form, including such properties as particle aggregation and flow. As will also be recognized, the preferred particle size for the agent used can be optimized by various means.

Therefore, a therapeutic agent used in the present invention is optionally encapsulated. Certain embodiments of the present invention include a composition wherein the therapeutic agent is dissolved, suspended or otherwise dispersed in a HA delivery vehicle.

Other embodiments of the present invention include a composition wherein particles of a therapeutic agent may be encapsulated in a micron and sub-micron form, wherein such a microparticle form is selected from a microsphere, a micropellet, a nanoparticle or a liposome.

Furthermore, the particles of unencapsulated agent or encapsulated agent may be suspended in a natural or synthetic biodegradable polymer or copolymer or blend thereof or adsorbed in a carrier matrix. Other equivalent unencapsulated or encapsulated forms are contemplated and fall with the scope of this invention. The unencapsulated or encapsulated agent or equivalent is then similarly dissolved or suspended in the HA delivery vehicle.

The microparticles form of the present invention may be prepared by any method known to those skilled in the art, which method is capable of producing such microparticles in a size range acceptable for use in an intra-articular injection formulations.

Several methods are commonly utilized for making a biodegradable polymeric microparticle form such as, but not limited to emulsion/solvent evaporation (also referred to as air extraction) and spray drying techniques.

The emulsion/solvent evaporation method includes the steps of:
1. Dissolving or dispersing one or more of a therapeutic agent, a biodegradable polymer or a blend thereof and other optional ingredients in an organic solvent or cosolvent to form an organic phase;
2. Mixing the organic phase with an aqueous phase to form an emulsion;
3. Extracting the emulsion into a larger volume of the organic phase solvent to thus form at least one microparticle; and
4. Recovering the microparticles using a filtration or centrifugation means and optionally washing the recovered microparticles.

The evaporation method includes process parameters such as solvent and anti-solvent selection, as well as temperature, stirring speed, and length of drying cycle which are tailorable by one skilled in the art to achieve optimal properties of the microparticles.

When using the evaporation method, optional ingredients, including surfactants (surface active agent) such as polyvinyl alcohol (PVA) may be incorporated to improve the compatibility of the therapeutic agent/polymer interface.

Suitable solvents for the polymer include organic solvents such as acetone; halogenated hydrocarbons such as chloroform and methylene; chloride aromatic hydrocarbon compounds, halogenated aromatic hydrocarbon compounds, cyclic ethers, alcohols and water. When the polymer is PLGA, a preferred solvent is methylene chloride.

The spray drying method includes the steps of:
1. Dissolving one or more of a therapeutic agent, a polymer and other optional ingredients in an organic solvent or cosolvent to form a homogeneous mixture; and
2. Spraying the mixture through one or more nozzles into a drying environment, wherein the environment is at an elevated temperature.

When using the spray drying method, optional surfactants such as Sodium Lauryl Sulfate (SLS), a Tween or PLURONIC may also be incorporated into the mixture to similarly improve the therapeutic agent/polymer interface.

Embodiments of the present invention include a form of the therapeutic agent selected from 1). a finely divided, uncoated particle, 2). a particle coated with a biodegradable polymer or a blend thereof, or 3). a particle coated with a biodegradable polymer or a blend thereof as a first layer and then with a plurality of biodegradable polymer layers or a blend thereof to thus form the microparticle.

Example of suitable polymers include, and are not otherwise limited to, poly-α-hydroxy acid polymers such as PLA (polylactic acid), PGA (polyglycolic acid); copolymers of PLA and PGA (PLGA) such as copolyoxalates, polycaprolactone and PLA-caprolactone or PLA-PLGA and others such as albumin, collagen, gelatin, HA, starch, cellulose and cellulose derivatives such as regenerated cellulose, methylcellulose, hydroxypropylcellulose, HPMC (hydroxypropylmethylcellulose), carboxymethylcellulose, cellulose acetate phthalate, cellulose acetate succinate or HPMC phthalate, casein, waxes such as glycerol mono- and di-stearate, dextrans, polysaccharides, fibrinogen, polyether esters, multiblock copolymers such as those based on PEG (polyethylene glycol) and polybutylene terephthalate, tyrosine-derived polycarbonates (e.g., see U.S. Pat. No. 6,120,491), polyhydroxyl acids, poly-D,L-lactides, poly-D,L-lactide-co-glycolide, polyglycolide, polyhydroxybutyrate, polydioxanone, polyalkyl carbonates and orthoesters, polyesters, poly-hydroxyvaleric acid, poly-malic acid, poly-tartronic acid, polyacrylamides, polyanhydrides, polyphosphazenes, poly-amino acids), poly-alkylene or oxide-polyester block copolymers such as X—Y, X—Y—X, Y—X—Y, R—(Y—X)ₙ, or R—(X—Y)ₙ,
wherein X is a polyalkylene oxide such as PEG, polypropylene glycol and block copolymers of polyethylene oxide and polypropylene oxide (e.g., PLURONIC and PLURONIC R series of polymers from BASF Corporation, Mount Olive, N.J.);
Y is a polyester, where the polyester may comprise the residues of one or more of the monomers selected from lactide, lactic acid, glycolide, glycolic acid, ε-caprolactone, γ-caprolactone, hydroxyvaleric acid, hydroxybutyric acid, β-butyrolactone, γ-butyrolactone, γ-valerolactone, γ-decanolactone, δ-decanolactone, trimethylene carbonate, 1,4-dioxane-2-one or 1,5-dioxepan-2-ones such as PLGA, PLA, PCL, polydioxanone and copolymers thereof, and
R is a multifunctional initiator and copolymers and blends thereof.

A polymer or copolymer or blend thereof for use in the present invention may be selected from a PLA polymer, a PLGA polymer or a PLA-PLGA polymer and the like or combinations thereof.

In an example of the present invention, a biodegradable microparticle is formed using a pharmaceutically acceptable biodegradable polymer selected from poly (α-hydroxy acid) polymers such as PLA, PGA and the PLGA copolymer of PLA and PGA.

The term “biodegradable” refers to the degradation mechanism for such polymers via hydrolysis into their monomer components.

Embodiments of the present invention include a hyaluronic acid delivery vehicle as the pharmaceutically acceptable carrier for the encapsulated therapeutic agent.

The suspending vehicle for a microparticle used in the composition of the present invention includes, but is not limited to, modified HA for intraarticular injection, collagen, polyalkylene oxide-based polymers, polysaccharides such as unmodified HA, chitosan and fucans, and copolymers of polysaccharides with degradable polymers.

Some examples of preferred polymeric carriers for the practice of this invention include polyethylene-co-vinyl acetate, polyurethanes, poly-D,L-lactic acid oligomers and polymers, poly-L-lactic acid oligomers and polymers, PGA, PLGA copolymers, copolymers of lactide and glycolide, polycaprolactone, polyvalerolactone, polyanhydrides, copolymers of polycaprolactone or PLA with a PEG such as MePEG or block copolymers such as X—Y, X—Y—X, Y—X—Y, R—(Y—X)ₙ, or R—(X—Y)ₙ, wherein X, Y and R are as previously defined.

An example of the HA used as the delivery vehicle of the present invention is HA or a salt form thereof. An embodiment of the present invention includes a pharmaceutical composition wherein the therapeutic agent is an optionally encapsulated NSAID selected from suprofen, tepoxalin or tolmetin and the like or a mixture thereof admixed in the HA delivery vehicle.

Another embodiment of the pharmaceutical composition includes an optionally encapsulated inflammatory kinase inhibitor or an isomer thereof as the therapeutic agent. An inflammatory kinase inhibitor for use in the present invention is a p38 MAP kinase inhibitor or an isomer thereof in an encapsulated form admixed in the HA delivery vehicle.

Another embodiment of the pharmaceutical composition includes an optionally encapsulated growth proliferation kinase inhibitor as the therapeutic agent. A growth proliferation kinase inhibitor for use in the present invention is sirolimus (rapamycin) in an encapsulated form admixed in the HA delivery vehicle.

Another embodiment of the pharmaceutical composition includes an optionally encapsulated inflammatory cytokine inhibitor as the therapeutic agent. An inflammatory cytokine inhibitor for use in the present invention is a TNF-α inhibitor, an IL-1α inhibitor, IL-1β inhibitor, IL-6 inhibitor, IL-10 inhibitor, IL-12 inhibitor or an IL-17 inhibitor in an encapsulated form admixed in the HA delivery vehicle.

Certain embodiments of the pharmaceutical composition include an optionally encapsulated inflammatory cytokine inhibitor selected from a TNF-α inhibitor, an IL-1α inhibitor, IL-1β inhibitor, IL-6 inhibitor or IL-10 inhibitor admixed in the HA delivery vehicle.

The pharmaceutical composition of the present invention is useful for treating a joint-capsule arthropathy when locally administered by intra-articular injection because an effective amount of at least one of a foregoing therapeutic agent is delivered directly to the site of inflammation.

The term “effective amount” means that amount of the optionally encapsulated therapeutic agent in the composition of the present invention that will result in improved healing, prevention, improvement treatment, or a decrease in the rate of advancement of a disease or disorder or amelioration of a disease, disorder, or side effect or will effectively enhance physiological function in a subject, animal or human, to thus treat, reduce or prevent the symptoms and to inhibit the development and progression of the joint-capsule arthropathy being treated.

The effective amount of an unencapsulated or encapsulated therapeutic agent for use in the present invention is from about 0.0001 mg/injection to about 300 mg/injection.

The present invention is also directed to a method for treating a joint-capsule arthropathy in a subject in need thereof comprising locally administering to the subject an effective amount of a pharmaceutical composition comprising an effective amount of one or more of an optionally encapsulated therapeutic agent in admixture with a hyaluronic acid delivery vehicle.

The method of locally administering the pharmaceutical composition of the present invention further comprises both transcapsular and transdiscal administration, whereby the composition is injected into the central joint space of the joint-capsule or the nucleus pulposus or annulus fibrosus of a disc, respectively.

“Transcapsular administration” means injecting the composition into the relatively intact joint space of a joint-capsule.

“Transdiscal administration” means injecting the composition into the relatively intact nucleus pulposus or annulus fibrosus spaces of a joint-capsule related to the spine.

“Subject” means an animal, preferably a mammal, most preferably a human, who is a patient or has been the object of treatment, observation or experiment and is at risk of (or susceptible to) developing a joint-capsule arthropathy or has already developed a joint-capsule arthropathy.

In embodiments of the method, an effective amount is a non-systemic, therapeutically effective amount.

In embodiments of the method, the non-systemic, therapeutically effective amount of an encapsulated or unencapsulated therapeutic agent is in a range of from about 0.0001 mg to about 300 mg.

In embodiments of the method, the joint-capsule arthropathy to be treated has symptoms of either periodic inflammation or chronic inflammation.

An embodiment of the method includes a non-systemic, therapeutically effective amount of a combination product for use in treating, ameliorating or preventing the development and progression of a joint-capsule arthropathy, wherein the composition comprises an effective amount of a combination selected from one or more of an optionally encapsulated therapeutic agent or one or more of an encapsulated therapeutic agent or a combination of two one or more an encapsulated therapeutic agent and one or more of an additional, different optionally encapsulated therapeutic agent.

An embodiment of the method for use of a combination product includes an effective amount of each ingredient for treating, ameliorating or preventing the development and progression of a joint-capsule arthropathy.

Pharmaceutical Composition

The HA delivery vehicle includes HA or a salt form thereof. A salt form of HA includes a sodium salt, potassium salt, ammonium salt or a C₍₁₋₅₎alkyl amine salt thereof. An embodiment of HA in the delivery vehicle is sodium hyaluronate, the sodium salt form of hyaluronic acid.

Embodiments of the present invention include HA or a salt form thereof having a Molecular Weight (MW) of from about 0.5 to about 9.0×10⁶ Daltons and is present at a concentration of from about 0.5% w/v to about 5.0% w/v.

An embodiment of the present invention includes a concentration of HA or salt form thereof in the range of from about 1% w/v to about 3% w/v.

An embodiment of the present invention includes a concentration of HA or salt form thereof in the range of from about 1.5% w/v to about 2.5% w/v.

An embodiment of the present invention includes a concentration of HA or salt form thereof of about 2% w/v.

The concentration of HA or salt form thereof used in the present invention may be varied depending on the MW of the HA used. As the MW of HA or salt form thereof is increased, the concentration would be proportionally decreased to maintain a HA MW to HA concentration ratio of from about 0.5% to about 5%. Conversely, as the MW of HA or salt form thereof is decreased, the concentration would be proportionally increased to maintain a concentration: MW ratio of from about 5% to about 0.5%.

The delivery vehicle has a pH in a range of from about 6.0 to about 8.0; or, a range of from about 7.0 to about 7.8; or, of about 7.4.

As compared to an isotonic saline solution of from about 0.8% to about 0.9%, the delivery vehicle has an osmotic pressure ratio of from about 0.7% to about 1.2%; or, a range of from about 0.8% to about 1.0%; or, a range of from about 0.85% to about 0.95%; or, of about 0.9% thereby yielding a preparation suitable for local administration to a joint-capsule.

A suitable aqueous solution of HA or salt form thereof has a viscosity in the range of from about 500 cps to about 2000 cps at a temperature of about 300° C.

The combination product is locally administered by intra-articular injection as a sterile formulation. Sterilization of the formulation may be accomplished in the usual ways, including aseptic preparation, filtration, exposure to gamma radiation, autoclaving, and the like.

Pharmaceutical Preparations

Representative pharmaceutical compositions of the present invention can be prepared in accordance with the methods described in the examples that follow below. The examples are offered by way of illustration, so the invention should not be construed as being limited by the materials or conditions used or composition forms prepared as expressed in the examples. Except where indicated, materials used in the examples were prepared by methodologies well known to persons of ordinary skill in the art. No attempt has been made to optimize the yields obtained in any of the examples. One skilled in the art would also know how to increase such yields through routine variations in ingredients or conditions and the like.

### EXAMPLE 1

A method of preparing a pharmaceutical composition comprising a therapeutic agent dispersed in an HA delivery vehicle is described.

The dose ranges were prepared by dilution to the final concentration. The therapeutic agent was sterilized via gamma irradiation (15 kgy) on dry ice.

Suprofen Dispersion

Suprofen was evenly dispersed in a commercially available form of purified HA (10 mL) and aseptically mixed.

Tolmetin Dispersion

Using the foregoing procedure, a dispersion containing tolmetin was similarly prepared.

Tepoxalin Dispersion

Tepoxalin (195 mg) was dissolved in DMSO (5 mL) and the solution was added to a commercially available form of purified HA (10 mL) and aseptically mixed.

Vehicle Control Formulation

Using the foregoing procedure, a vehicle control formulation was similarly prepared without the agent.

### EXAMPLE 2

The dispersion formulations prepared in Example 1 were used to prepare three dose levels for each formulation by dilution with a commercially available form of purified HA. Aliquots of each diluted formulation were then dispensed (250 μL) into syringes (1 mL) for use in Example 4.

Suprofen Formulation

The suprofen formulation from Example 1 was aseptically, evenly mixed in the commercially available form of purified HA to provide a 13.3 μg/μL stock solution (high dose). A middle 1.33 μg/μL dose and a low 0.133 μg/μL dose were prepared by dilution with the purified HA.

Tolmetin Formulation

Using the procedure for preparation of the suprofen formulation, a 13.3 μg/μL high dose, a 1.33 μg/μL middle dose and a 0.133 μg/μL low dose for the tolmetin formulation from Example 1 were also prepared by dilution.

Tepoxalin Formulation

Using the procedure for preparation of the suprofen formulation, a 12.6 μg/μL high dose, a 1.26 μg/μL middle dose and a 0.126 μg/μL low dose for the tepoxalin formulation from Example 1 were also prepared by dilution.

Vehicle Control Formulation

Using the procedure described for dilution of the suprofen formulation dose levels, the vehicle control formulation from Example 1 was similarly prepared without the agent.

### EXAMPLE 3

Stability Testing:

The stability of a pharmaceutical composition comprising a therapeutic agent dispersed in an HA delivery vehicle at a high, middle and low concentration while stored at 4° C. was tested over a 4 month period.

All concentrations were run in duplicate by dissolving the samples in DMSO (10 mL). The samples were injected on an HPLC against a known reference standard. The average percent drug recovery at the start of the study (T₀) and at the one month and 4 month timepoints are shown in Table 1.

TABLE 1
Average Content Recovery

| | | Concentration | | | |
|---|---|---|---|---|---|
| Agent | Dosage | (μg/μL) | T₀ | 1 month | 4 month |
| Suprofen | High | 13.3 | 97.6 | 98.4 | 95.3 |
| | Mid | 1.33 | 98.4 | 101.7 | 97.0 |
| | Low | 0.133 | 96.6 | 96.8 | 94.5 |
| Tepoxalin | High | 13.3 | 98.6 | 99.4 | 95.3 |
| | Mid | 1.33 | 97.3 | 96.7 | 94.9 |
| | Low | 0.133 | 98.7 | 103.4 | 96.7 |
| Tolmetin | High | 12.6 | 97.3 | 95.6 | 97.6 |
| | Mid | 1.26 | 99.8 | 98.7 | 102.8 |
| | Low | 0.126 | 106.4 | 99.6 | 110.5 |

### EXAMPLE 4

Rabbit Anterior Cruciate Ligament Transection (A CLT) Model

Osteoarthritis is a degenerative disease that degrades cartilage, resulting in pain, restriction of motion and deformity. In the ACLT model, the appearance of OA can be seen at 6 weeks and is characterized by erosion of cartilage on the trochlear grove and mild to serious cartilage erosion on the femoral condyle. The last symptoms to arise are on the tibial plateau. At 6 weeks, there is some damage visible on the tibial plateau, but the damage may not be great given the length of the study. Osteophyte formation is usually how disease onset is first established, but ACLT model rabbits form osteophytes readily (some joints without surgery may demonstrate osteophyte formation). While osteophyte formation is a factor in the analysis, several parameters are observed, wherein each parameter alone is not sufficient to draw a conclusion. Therefore, the composite score provides a more meaningful result for a particular agent tested.

The ACLT model was used to evaluate the ability of a NSAID agent in a pharmaceutical formulation administered directly into the intra-articular capsule of the knee to reduce the incidence of osteoarthritic changes. The NSAIDs formulations prepared in Example 2 were used and injected 6 weeks after animal preparation. A commercially available form of purified HA was used as the vehicle control. The animals (n=7 per group) were dosed (160 μL per injection) once per week for 5 weeks.

Table 2 lists the IC₅₀ for each NSAID formulation used.

Animals (Housing and Care and Preparation):

The animals were group housed in pens. Diet consisted of a commercially available rabbit chow and tap water. The animals were handled and maintained in accordance with applicable humane requirements.

Female New Zealand White Rabbits (n=84, Milibrook Breeding Labs, SPF) used in the study underwent ACLT on the right knee.

One week after the last injection (total 12 weeks from time of ACLT), animals were euthanized and gross observations were made on the knee joints. Histological sectioning and staining were performed on the condyles.

Analysis and Results

The joints were analyzed for disease state in the trochlear groove, the femoral condyles and the tibial plateau. Pathological assessments were performed on the sectioned tissue. Condylar structure, cellularity, presence of GAG (glycosaminoglycans) and presence of cartilage were evaluated.

On the anterior surface of the femur (trochlear groove), the number of osteophytes, the size (diameter) of osteophytes, the presence of trochlear groove thickening and the erosion of cartilage were evaluated grossly.

On the femoral condyles, the erosion of cartilage (% surface area) (both medial and lateral condyle), the erosion of cartilage (depth) and the presence of clefts were evaluated grossly.

On the tibial plateau, observations on the thickening, presence of clefts and erosion were made.

Each parameter was given a score. The combination of all parameters gave a Total Score. A higher score indicates more damage to the joint. The data for un-operated joints (no surgery) has been included as a baseline for a normal joint.

Trochlear Groove (Erosion of Cartilage):

Erosion of cartilage was graded. All suprofen dose levels and the high doses of tepoxalin and tolmetin were numerically lower than the Vehicle control and No Injection treated groups.

Femoral Condyle (% Surface Area Erosion):

Percent surface area erosion was graded. On the medial femoral condyle the mid dose group of suprofen, all dose groups of tepoxalin and the low and mid dose groups of tolmetin had average numerical scores less than Vehicle and No Injection treated groups. The low dose of suprofen demonstrated the same average score as No Injection but scored better than Vehicle.

On the lateral femoral condyle the mid and high dose group of suprofen and the mid doses of both tepoxalin and tolmetin had average numerical scores less than Vehicle and No Injection treated groups.

Femoral Condyle (Depth of Erosion):

Depth of cartilage erosion on the femoral condyle was graded. All treatment groups, except the high dose group of tolmetin, demonstrated a numerically lower average score compared to No Injection. The high dose group of tolmetin had the same average score as the No Injection group. All treatment groups except the high doge groups of tepoxalin and tolmetin demonstrated lower average mean scores than the Vehicle treated group.

All Trochlear Groove Effects:

The score for all trochlear groove effects is a composite of grades from all criteria within this category. The grades that compose this parameter are the criteria listed for the Anterior Surface of Femur (Trochlear Groove). All suprofen treated groups and the high dose groups of tepoxalin and tolmetin demonstrated lower mean scores than Vehicle and No Injection treated groups.

All Condylar Effects:

The score for all condylar effects is a composite of grades from all criteria within this category. The grades that compose this parameter are the criteria listed for Femoral Condyles. All groups, except the tolmetin high dose group demonstrated a lower average score for all condylar effects than the Vehicle or No Injection treated groups.

All Tibial Plateau Effects:

The score for all tibial plateau effects is a composite of grades from all criteria within this category. The grades that compose this parameter are the criteria for Tibial Plateau. All treatment groups, except the low dose of suprofen and the high dose of tolmetin, demonstrated lower average scores than the Vehicle treated group. The mid doses of suprofen and tolmetin demonstrated lower average scores than the No Injection treated group.

All Cartilage Effects:

The score for all cartilage effects is a composite of grades from all criteria within this category. The grades that compose this parameter are trochlear grove thickness and erosion, all the grades in all condylar effects, and all grades listed under tibial plateau. All treatment groups, except the high dose of tolmetin, demonstrated lower mean scores for all cartilage effects than Vehicle. The mid and high dose groups of suprofen and tepoxalin and the mid dose group of tolmetin demonstrated lower mean scores than the No Injection treated group.

Total Score:

The composite score is a total of grades from all of the scoring criteria. All groups, except the mid dose of tepoxalin, demonstrated numerically less average scores than the Vehicle treated group. The mid and high dose groups of suprofen, the low and high dose groups of tepoxalin and the mid dose group of tolmetin demonstrated numerically less average scores than the No Injection treated group.

TABLE 2

| Drug | In Vitro Efficacy | IC₅₀ | |
|---|---|---|---|
| Suprofen | Inhibitor of 5LO-1 | 3 | μM |
| | Inhibitor of COX 1 | 60 | nM |
| | Inhibitor of COX 2 | 1 | μM |
| Tepoxalin | Inhibitor of 5LO-1 | 170 | nM |
| | Inhibitor of COX-1 | 970 | nM |
| | Inhibitor of COX-2 | 2 | uM |
| | Inhibitor of LTB₄ | 40 | nM |
| | Inhibitor of TXB₂ | 2 | nM |
| Tolmetin | Inhibitor or 5LO-1 | 30 | nM |
| | Inhibitor of COX-1 | 3 | μM |
| | Inhibitor of COX-2 | 50 | nM |

### EXAMPLE 5

A method of preparing a pharmaceutical composition comprising an encapsulated NSAID suprofen in a microsphere (MP) form incorporated in an HA delivery vehicle is described. The dose ranges were prepared by dilution to the final concentration.

Suprofen MP

Suprofen (0.2 g) and PLGA (1.8 g) (75/25 ratio) were dissolved in methylene chloride (6.2 g). The suprofen/PLGA solution was added to an aqueous solution of polyvinyl alcohol (PVA) (155 g, 2% w/v) and homogenized for approximately 1 minute to form an emulsion. Demineralized water (155 g) was added to dilute the emulsion and then the mixture was stirred for 3 hours. The microspheres were collected, dried under vacuum and sterilized by gamma irradiation (15 kg ray) on dry ice.

MP Formulation A

Five (5) sets suprofen MP (80 mg each) were aseptically mixed in a commercially available form of purified HA (6 mL).

MP Formulation A Control

Five (5) sets of a control formulation for MP Formulation A were similarly prepared without the agent.

MP Formulation B

One (1) set suprofen MP (400 mg) was aseptically mixed in a commercially available form of purified HA (30 mL).

MP Formulation B Control

One (1) set of a control formulation for MP Formulation B was similarly prepared without the agent.

An aliquot volume of 250 μL from each formulation was then dispensed into syringes (1 mL) for use in Example 7.

### EXAMPLE 6

A method of preparing a pharmaceutical composition comprising the NSAID suprofen in a block co-polymer (BCP) admixed in an HA delivery vehicle is described.

BCP Active Formulation A and B

An active formulation containing suprofen (0.5% by wt) was mixed with a PCL-GA-LA polymer (MW 12,400, 35% by wt) in benzyl benzoate (65% by wt) at room temperature using an IKA electric stirrer at a setting of between 50-300 rpm.

An aliquot volume of 250 μL from the BCP Active Formulation A was then dispensed into five (5) syringes (1 mL) for use in Example 7.

An aliquot volume of 250 μL from the BCP Active Formulation B was then dispensed into one (1) syringe (1 mL) for use in Example 7.

BCP Control Formulation A & B

A control formulation for each BCP active formulation was similarly prepared without the agent.

An aliquot volume of 250 μL from the BCP Control Formulation A was then dispensed into five (5) syringes (1 mL) for use in Example 7.

An aliquot volume of 250 μL from the BCP Control Formulation B was then dispensed into one (1) syringe (1 mL) for use in Example 7.

### EXAMPLE 7

Using the ACLT model of Example 4, the formulations of Example 5 and Example 6 were compared against a vehicle formulation and to a control group which received no injection. Scores are expressed using the criteria of Example 4 as Score (±SEM). Medial Femoral Condyle (% surface area erosion):

The MP Formulation A Score was 0.73 (±0.27) compared to MP Formulation A Control 0.17 (±0.11).

The BCP Formulation A Score was 1.0 (±0.43) compared to BCP Formulation A Control 0.5 (±0.29).

The MP Formulation B Score was 0.92 (±0.43) compared to MP Formulation B Control 0.27 (±0.14).

The BCP Formulation B Score was 0.5 (±0.29) compared to BCP Formulation B Control 0.45 (±0.21).

The No Injection Score was 0.58 (±0.34).

Lateral Femoral Condyle (% Surface Area Erosion):

The MP Formulation A Score was 0.73 (±0.43) compared to MP Formulation A Control 0.17 (±0.11).

The BCP Formulation A Score was 0.58 (±0.36) compared to BCP Formulation A Control 1.33 (±0.4).

The MP Formulation B Score was 1.45 (±0.47) compared to MP Formulation B Control 0.82 (±0.33).

The BCP Formulation B Score was 1.58 (±0.45) compared to BCP Formulation B Control 0.82 (±0.35).

The No Injection Score was 0.33 (±0.33).

All Condylar Effects:

The all condylar effects score is a composite of the medial and femoral condyle grades.

The MP Formulation A Score was 2.5 (±1.15) compared to MP Formulation A Control 0.5 (±0.21).

The BCP Formulation A Score was 3.0 (±1.18) compared to BCP Formulation A Control 3.42 (±1.1).

The MP Formulation B Score was 4.59 (±1.72) compared to MP Formulation B Control 2.41 (±0.71).

The BCP Formulation B Score was 3.63 (±1.18) compared to BCP Formulation B Control 2.14 (±0.77).

The No Injection Score was 1.38 (±0.66).

It is to be understood that the preceding description teaches the principles of the present invention, with examples thereof which have emphasized certain aspects. It will also be understood that the practice of the invention encompasses all of the usual variations, adaptations and modifications as come within the scope of the following claims and their equivalents. However, numerous other equivalents not specifically elaborated on or discussed may nevertheless fall within the spirit and scope of the present invention and claims and are intended to be included.

Throughout this application, various publications are cited. The disclosure of these publications is hereby incorporated by reference into this application to describe more fully the state of the art to which this invention pertains.

What is claimed is:

1. A pharmaceutical composition for use in treating a joint-capsule arthropathy in a subject in need thereof comprising an effective amount of one or more of a locally administered, optionally encapsulated therapeutic agent in admixture with a hyaluronic acid delivery vehicle.

2. The composition of claim 1, wherein the joint-capsule arthropathy is selected from osteoarthritis, joint-capsule injuries which have triggered or will trigger a pro-inflammatory response or arthropathies which have triggered or will trigger a pro-inflammatory response.

3. The composition of claim 2, wherein the joint-capsule arthropathy is selected from a symptomatic range of arthropathies that are just beginning to develop, wherein bone degeneration and the level of pain experienced by the subject is at a minimum to those arthropathies wherein bone degeneration and the level of pain experienced by the subject requires surgical intervention or where the administration of one or more of a therapeutic agent for pain intervention has led to a decreased quality of life.

4. The composition of claim 3, wherein joint-capsule injuries are the result of an endogenous or exogenous source of insult.

5. The composition of claim 4, wherein the endogenous source of insult is a genetic joint-disorder, abnormal bone growth, aggregation or cyst formation in a joint space, joint fusion, joint misalignment, compensation for joint misalignment or other types of musculo-skeletal injuries in other parts of the body or injuries resulting from wear, repetitive motion or immobility.

6. The composition of claim 4, wherein the exogenous source of insult is a musculo-skeletal injury caused by trauma, a sport-related injury or immobility.

7. The composition of claim 1, wherein at least one therapeutic agent is encapsulated.

8. The composition of claim 1, further comprising one or more of an encapsulated therapeutic agent and one or more of an additional, different optionally encapsulated therapeutic agent.

9. The composition of claim 1, further comprising one or more of an encapsulated therapeutic agent and one or more of an additional, different unencapsulated therapeutic agent.

10. The composition of claim 1, further comprising two or more of an encapsulated therapeutic agent, wherein each encapsulated agent has a dissimilar release profile.

11. The composition of claim 1, further comprising two or more of an encapsulated therapeutic agent, each having a dissimilar release profile, and one or more of an optionally encapsulated therapeutic agent.

12. The composition of claim 1, wherein the therapeutic agent is selected from a non-steroidal anti-inflammatory drug, a nitric oxide inhibitor, a cyclooxygenase inhibitor, a prostaglandin inhibitor, an interleukin inhibitor, a leukotriene inhibitor, an inflammatory or growth proliferation kinase inhibitor, an inflammatory cytokine inhibitor, a corticosteroid, a hyaluronidase enzyme inhibitor, a matrix metalloproteinase inhibitor, an aggrecanase inhibitor, an apoptosis inhibitor, a cartilage enhancing factor or a bone morphogenic protein.

13. The composition of claim 12, wherein the non-steroidal anti-inflammatory drug is selected from analogs of carboxylic acid, acetic acid, propionic acid, salicylic acid, hydratropic acid, hydroxamic acid, benzoic/xylylanthranilic acid, benzene acetic acid, indole acetic acid, indene acetic acid, toluoylpyrrole acetic acid, naphthalene acetic acid, benzopyranopyridine acetic acid, pyrazolidinedione or benzthiazine and the like or a salt or ester form or mixture thereof.

14. The composition of claim 12, wherein the non-steroidal anti-inflammatory drug is selected from suprofen, tepoxalin, mefenamic acid, ibuprofen, diclofenac, alclofenac, indomethacin, sulindac, tolmetin, naproxen, pranoprofen, phenylbutazone, oxyphenbutazone or piroxicam and the like or a salt or ester form or mixture thereof.

15. The composition of claim 14, wherein the non-steroidal anti-inflammatory drug is selected from suprofen, tepoxalin, ibuprofen, diclofenac, indomethacin, sulindac, tolmetin, naproxen or oxyphenbutazone and the like or a salt or ester form or mixture thereof.

16. The composition of claim 15, wherein the non-steroidal anti-inflammatory drug is selected from suprofen, tepoxalin or tolmetin and the like or a salt or ester form or mixture thereof.

17. The composition of claim 12, wherein the therapeutic agent is at least one non-steroidal anti-inflammatory drug which is a cyclooxygenase, leukotriene or prostaglandin inhibitor.

18. The composition of claim 17, wherein the cyclooxygenase inhibitor is a 5LO-1, COX-1 or COX-2 inhibitor.

19. The composition of claim 17, wherein the leukotriene inhibitor is a LTB₄ inhibitor.

20. The composition of claim 17, wherein the prostaglandin inhibitor is a PGE₂ inhibitor.

21. The composition of claim 13, wherein the non-steroidal anti-inflammatory drug is a 5LO-1, COX-1, COX-2, LTB₄ or TXB₂ inhibitor.

22. The composition of claim 21, wherein the 5LO-1 inhibitor is an inhibitor having an IC₅₀ of from about 0.010 μM to about 10 μM.

23. The composition of claim 21, wherein the COX-1 inhibitor is an inhibitor having an IC₅₀ of from about 0.06 μM to about 12 μM.

24. The composition of claim 21, wherein the COX-2 inhibitor is an inhibitor having an IC₅₀ of from about 0.05 μM to about 6 μM.

25. The composition of claim 21, wherein the LTB₄ inhibitor is an inhibitor having an IC₅₀ of from about 0.035 μM to about 2 μM.

26. The composition of claim 21, wherein the TXB₂ inhibitor is an inhibitor having an IC₅₀ of from about 0.002 μM to about 0.04 μM.

27. The composition of claim 12, wherein the inflammatory kinase inhibitor is a MAP kinase inhibitor or isoform thereof.

28. The composition of claim 27, wherein the MAP kinase inhibitor is a p38, ERK or JNK2 kinase inhibitor or isoform thereof.

29. The composition of claim 28, wherein the isoform of the p38 MAP kinase inhibitor is a selective p38α MAP kinase inhibitor.

30. The composition of claim 12, wherein the growth proliferation kinase inhibitor is sirolimus.

31. The composition of claim 12, wherein the inflammatory cytokine inhibitor is selected from a TNF-α inhibitor, an IL-1α inhibitor, IL-1β inhibitor, IL-6 inhibitor, IL-10 inhibitor, IL-12 inhibitor or an IL-17 inhibitor.

32. The composition of claim 31, wherein the inflammatory cytokine inhibitor is selected from a TNF-α inhibitor, an IL-1α inhibitor, IL-1β inhibitor, IL-6 inhibitor or IL-10 inhibitor.

33. The composition of claim 1, wherein the therapeutic agent is one or more of a non-steroidal anti-inflammatory drug selected from claim 13.

34. The composition of claim 1, wherein the therapeutic agent is one or more of a non-steroidal anti-inflammatory drug selected from claim 13 in combination with one or more of an additional, different therapeutic agent selected from claim 12.

35. The composition of claim 1, wherein the composition further comprises a combination product having two or more of a therapeutic agent selected from claim 12.

36. The composition of claim 35, wherein the combination product further comprises one or more of a therapeutic agent selected from claim 12 and at least one additional, different therapeutic agent selected from claim 12.

37. The composition of claim 35, wherein the combination product further comprises a mixture of two or more of a therapeutic agent selected from claim 12 for use in treating a joint-capsule arthropathy, wherein the combination product achieves pain relief, modification of the arthropathy and its progression or delay of surgical intervention.

38. The composition of claim 1, wherein the therapeutic agent further comprises a pharmaceutically acceptable salt, pro-drug or pharmaceutically active metabolite of a therapeutic agent selected from claim 12.

39. The composition of claim 1, wherein the therapeutic agent is present in a particle form, wherein the particle form is selected from a finely-divided particle, wherein the particle size is in a range of from about 0.5 microns to about 200 microns.

40. The composition of claim 39, wherein the particle size is in a range of from about 5 microns to about 50 microns.

41. The composition of claim 39, wherein the mean particle size is in a range of from about 25 microns to about 50 microns.

42. The composition of claim 1, wherein the therapeutic agent is dissolved, suspended or otherwise dispersed in the hyaluronic acid delivery vehicle.

43. The composition of claim 42, wherein the therapeutic agent is suspended in a natural or synthetic biodegradable polymer or copolymer or mixture thereof or adsorbed in a carrier matrix.

44. The composition of claim 1, wherein the encapsulated form of the therapeutic agent is a microparticle form selected from a microsphere, a micropellet, a nanoparticle, or a liposome.

45. The composition of claim 44, wherein the microparticle form is prepared by the steps of
(a) dissolving or dispersing one or more of a therapeutic agent, a biodegradable polymer or a blend thereof and other optional ingredients in an organic solvent or cosolvent to form an organic phase;
(b) mixing the organic phase from step (a) with an aqueous phase to form an emulsion; (c) extracting the emulsion from step (b) into a larger volume of the organic phase solvent to thus form at least one microparticle; and
(d) recovering the microparticle from step (c) using a filtration or centrifugation means and optionally washing the recovered microparticles.

46. The composition of claim 1, wherein the therapeutic agent is selected from a finely-divided uncoated particle, a particle coated with a biodegradable polymer or a blend thereof or a particle coated with a biodegradable polymer or a blend thereof as a first layer and then with a plurality of biodegradable polymer layers or a blend thereof.

47. The composition of claim 46, wherein the polymer or copolymer or blend thereof is selected from PLA, PGA, PLGA or PLA-PLGA or a blend thereof.

48. The composition of claim 38, wherein the carrier matrix is hyaluronic acid.

49. The composition of claim 1, wherein the hyaluronic acid delivery vehicle is a purified form of hyaluronic acid or a salt form thereof.

50. The composition of claim 49, wherein the hyaluronic acid salt form is a sodium salt, potassium salt, ammonium salt or a C₍₁₋₅₎alkyl amine salt thereof.

51. The composition of claim 49, wherein the purified salt form of hyaluronic acid is sodium hyaluronate.

52. The composition of claim 49, wherein the hyaluronic acid has a Molecular Weight of from about 0.5 to about 6.0×10⁶ Daltons and is present at a concentration of from about 0.5% w/v to about 2% w/v.

53. The composition of claim 49, wherein the hyaluronic acid is present at a concentration in a range of from about 1% w/v to about 2% w/v.

54. The composition of claim 1, wherein the hyaluronic acid delivery vehicle has a pH of from about 6.0 to about 7.0.

55. The composition of claim 1, wherein the hyaluronic acid delivery vehicle has an osmotic pressure ratio of from about 0.8 to about 1.2 as compared to an isotonic saline solution of from about 0.8% to about 0.9%.

56. The composition of claim 1, wherein the hyaluronic acid delivery vehicle has a viscosity in the range of from about 500 cps to about 2000 cps at a temperature of about 300° C.

57. The composition of claim 1, wherein the therapeutic agent is an optionally encapsulated non-steroidal anti-inflammatory drug selected from suprofen, tepoxalin or tolmetin or a mixture thereof.

58. The composition of claim 1, wherein the therapeutic agent is an optionally encapsulated inflammatory kinase inhibitor selected from a p38 MAP kinase inhibitor or an isoform thereof.

59. The composition of claim 1, wherein the therapeutic agent is an optionally encapsulated growth proliferation kinase inhibitor selected from sirolimus.

60. The composition of claim 1, wherein the therapeutic agent is an optionally encapsulated inflammatory cytokine inhibitor selected from a TNF-α inhibitor, an IL-1α inhibitor, IL-1βinhibitor, IL-6 inhibitor, IL-10 inhibitor, IL-12 inhibitor or an IL-17 inhibitor.

61. The composition of claim 1, wherein the therapeutic agent is an optionally encapsulated inflammatory cytokine inhibitor selected from a TNF-α inhibitor, an IL-1α inhibitor, IL-1β inhibitor, IL-6 inhibitor or IL-10 inhibitor.

62. The composition of claim 1, wherein the effective amount of an unencapsulated therapeutic agent is from about 0.0001 mg/injection to about 300 mg/injection.

63. The composition of claim 1, wherein the effective amount of an encapsulated therapeutic agent is from about 0.0001 mg/injection to about 300 mg/injection.

64. A method for treating a joint-capsule arthropathy in a subject in need thereof comprising locally administering to the subject an effective amount of a pharmaceutical composition comprising an effective amount of an optionally encapsulated therapeutic agent in admixture with a hyaluronic acid delivery vehicle.

65. The method of claim 64, wherein the joint-capsule arthropathy is selected from osteoarthritis, joint-capsule injuries which have triggered or will trigger a pro-inflammatory response or arthropathies which have triggered or will trigger a pro-inflammatory response.

66. The method of claim 65, wherein the joint-capsule arthropathy is selected from a symptomatic range of arthropathies that are just beginning to develop, wherein bone degeneration and the level of pain experienced by the subject is at a minimum to those arthropathies wherein bone degeneration and the level of pain experienced by the subject requires surgical intervention or where the administration of one or more of a therapeutic agent for pain intervention has led to a decreased quality of life.

67. The method of claim 66, wherein joint-capsule injuries are the result of an endogenous or exogenous source of insult.

68. The method of claim 67, wherein the endogenous source of insult is a genetic joint-disorder, abnormal bone growth, aggregation or cyst formation in a joint space, joint fusion, joint misalignment, compensation for joint misalignment or other types of musculo-skeletal injuries in other parts of the body or injuries resulting from wear, repetitive motion or immobility.

69. The method of claim 67, wherein the exogenous source of insult is a musculo-skeletal injury caused by trauma, a sport-related injury or immobility.

70. The method of claim 64, wherein the therapeutic agent is selected from a non-steroidal anti-inflammatory drug, a nitric oxide inhibitor, a cyclooxygenase inhibitor, a prostaglandin inhibitor, an interleukin inhibitor, a leukotriene inhibitor, an inflammatory or growth proliferation kinase inhibitor, an inflammatory cytokine inhibitor, a corticosteroid, a hyaluronidase enzyme inhibitor, a matrix metalloproteinase inhibitor, an aggrecanase inhibitor, an apoptosis inhibitor, a cartilage enhancing factor or a bone morphogenic protein.

71. The method of claim 70, wherein the non-steroidal anti-inflammatory drug is selected from analogs of carboxylic acid, acetic acid, propionic acid, salicylic acid, hydratropic acid, hydroxamic acid, benzoic/xylylanthranilic acid, benzene acetic acid, indole acetic acid, indene acetic acid, toluoylpyrrole acetic acid, naphthalene acetic acid, benzopyranopyridine acetic acid, pyrazolidinedione or benzthiazine and the like or a salt or ester form or mixture thereof.

72. The method of claim 71, wherein the non-steroidal anti-inflammatory drug is selected from suprofen, tepoxalin, mefenamic acid, ibuprofen, diclofenac, alclofenac, indomethacin, sulindac, tolmetin, naproxen, pranoprofen, phenylbutazone, oxyphenbutazone or piroxicam and the like or a salt or ester form or mixture thereof.

73. The method of claim 72, wherein the non-steroidal anti-inflammatory drug is selected from suprofen, tepoxalin, ibuprofen, diclofenac, indomethacin, sulindac, tolmetin, naproxen or oxyphenbutazone and the like or a salt or ester form or mixture thereof.

74. The method of claim 73, wherein the non-steroidal anti-inflammatory drug is selected from suprofen, tepoxalin or tolmetin and the like or a salt or ester form or mixture thereof.

75. The method of claim 64, wherein the therapeutic agent is at least one non-steroidal anti-inflammatory drug which is a cyclooxygenase, leukotriene or prostaglandin inhibitor.

76. The method of claim 75, wherein the cyclooxygenase inhibitor is a 5LO-1, COX-1 or COX-2 inhibitor.

77. The method of claim 75, wherein the leukotriene inhibitor is a LTB₄ inhibitor.

78. The method of claim 75, wherein the prostaglandin inhibitor is a PGE₂ inhibitor.

79. The method of claim 71, wherein the non-steroidal anti-inflammatory drug is a 5LO-1, COX-1, COX-2, LTB₄ or TXB₂ inhibitor.

80. The method of claim 79, wherein the 5LO-1 inhibitor is an inhibitor having an IC₅₀ of from about 0.010 μM to about 10 μM.

81. The method of claim 79, wherein the COX-1 inhibitor is an inhibitor having an IC₅₀ of from about 0.06 μM to about 12 μM.

82. The method of claim 79, wherein the COX-2 inhibitor is an inhibitor having an IC₅₀ of from about 0.05 μM to about 6 μM.

83. The method of claim 79, wherein the LTB₄ inhibitor is an inhibitor having an IC₅₀ of from about 0.035 μM to about 2 μM.

84. The method of claim 79, wherein the TXB₂ inhibitor is an inhibitor having an IC₅₀ of from about 0.002 μM to about 0.04 μM.

85. The method of claim 70, wherein the inflammatory kinase inhibitor is a MAP kinase inhibitor or isoform thereof.

86. The method of claim 85, wherein the MAP kinase inhibitor is a p38, ERK or JNK2 kinase inhibitor or isoform thereof.

87. The composition of claim 86, wherein the isoform of the p38 MAP kinase inhibitor is a selective p38α MAP kinase inhibitor.

88. The method of claim 70, wherein the growth proliferation kinase inhibitor is sirolimus.

89. The method of claim 70, wherein the inflammatory cytokine inhibitor is selected from a TNF-α inhibitor, an IL-1α inhibitor, IL-1β inhibitor, IL-6 inhibitor, IL-10 inhibitor, IL-12 inhibitor or an IL-17 inhibitor.

90. The method of claim 89, wherein the inflammatory cytokine inhibitor is selected from a TNF-α inhibitor, an IL-1α inhibitor, IL-1β inhibitor, IL-6 inhibitor or IL-10 inhibitor.

91. The method of claim 64, wherein the therapeutic agent is one or more of a non-steroidal anti-inflammatory drug selected from claim 71.

92. The method of claim 64, wherein the therapeutic agent is one or more of a non-steroidal anti-inflammatory drug selected from claim 71 in combination with one or more of an additional, different therapeutic agent selected from claim 70.

93. The method of claim 64, wherein the composition further comprises a combination product having two or more of a therapeutic agent selected from claim 70.

94. The method of claim 93, wherein the combination product further comprises one or more of a therapeutic agent selected from claim 70 and at least one additional, different therapeutic agent selected from claim 70.

95. The method of claim 64, wherein the combination product further comprises a mixture of two or more of a therapeutic agent selected from claim 70 for use in treating a joint-capsule arthropathy, wherein the combination product achieves pain relief, modification of the arthropathy and its progression or delay of surgical intervention.

96. The method of claim 64, wherein the composition further comprises a pharmaceutically acceptable salt, pro-drug or pharmaceutically active metabolite of a therapeutic agent selected from claim 70 in admixture with a pharmaceutically acceptable carrier.

97. The method of claim 64, wherein the therapeutic agent is present in a particle form, wherein the particle form is selected from a finely-divided particle, wherein the particle size is in a range of from about 0.5 microns to about 200 microns.

98. The method of claim 97, wherein the particle size is in a range of from about 5 microns to about 50 microns.

99. The method of claim 97, wherein the mean particle size is in a range of from about 25 microns to about 50 microns.

100. The method of claim 64, wherein the therapeutic agent is dissolved, suspended or otherwise dispersed in the hyaluronic acid delivery vehicle.

101. The method of claim 64, wherein the therapeutic agent is suspended in a natural or synthetic biodegradable polymer or copolymer or mixture thereof or adsorbed in a carrier matrix.

102. The method of claim 64, wherein the encapsulated form of the therapeutic agent is a microparticle form selected from a microsphere, a micropellet, a nanoparticle, or a liposome.

103. The method of claim 64, wherein the effective amount is a non-systemic, therapeutically effective amount.

104. The method of claim 64, wherein the effective amount is in a range of from about 0.0001 mg/injection to about 300 mg/injection.

105. The method of claim 64, wherein the method of locally administering further comprises transcapsular or transdiscal administration.

106. The method of claim 64, wherein the joint capsule having an arthropathy is selected from a joint capsule having symptoms of periodic inflammation or a joint capsule having symptoms of chronic inflammation.

107. The method of claim 64, wherein the method further comprises a non-systemic, therapeutically effective amount of a combination product for use in treating, ameliorating or preventing the development and progression of a joint-capsule arthropathy.

108. The method of claim 107, wherein the combination product comprises a combination selected from one or more of an optionally encapsulated therapeutic agent or one or more of an encapsulated therapeutic agent or a combination of two one or more an encapsulated therapeutic agent and one or more of an additional, different optionally encapsulated therapeutic agent, wherein one or more of the therapeutic agent is each selected from claim 70.
